(19) United States
(12) Patent Application Publication
(10) Pub. No.: US 2007/0086639 A1
(43) Pub. Date: Apr. 19, 2007

(54) Apparatus, method, and program for image processing

(75) Inventor: Hideyuki Sakaida, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation

(21) Appl. No.: 11/546,999

(22) Filed: Oct. 13, 2006

(30) Foreign Application Priority Data
Oct. 13, 2005 (JP) 298330/2005
Oct. 13, 2005 (JP) 298331/2005
Oct. 13, 2005 (JP) 298332/2005

(51) Int. Cl. G06K 9/00; A61B 5/00

(52) U.S. Cl. 382/132; 600/300

(57) ABSTRACT

A rib image is inferred accurately from a chest image. Rib images are generated by extraction of pixel value components contributing to ribs from values of pixels comprising respective chest images, and rib overlaps at which ribs appear to overlap are detected in the respective rib images. The rib images are normalized so as to cause positions of the rib overlaps detected in the chest images to agree with each other, and the pixel values of the rib images are analyzed by using a statistical method on the normalized rib images. By using a result of the analysis, an inferred rib image is generated by inferring pixel values of a rib region in a chest image of a predetermined subject.

### BACKGROUND OF THE INVENTION

1. Field of the Invention

The present invention relates to an image processing apparatus and an image processing method for generating a rib image from a chest image. The present invention also relates to a program that causes a computer to execute the image processing method.

2. Description of the Related Art

In the field of medicine, CAD (Computer Aided Diagnosis) apparatuses have been provided for automatically detecting abnormal shadows in digital medical images by use of computers. One of such apparatuses is a chest CAD apparatus for detecting a shadow of tumor in a digital chest X-ray image.

Chest X-ray images include so-called “background images” that are images representing structures of various anatomical characteristics such as ribs and clavicles. The background images disrupt detection of an abnormal shadow, and causes deterioration in detection performance. Therefore, a method has been proposed for chest CAD processing by removing such a background image through filtering processing (see U.S. Pat. No. 5,289,374, for example).

Since the anatomical structures of the chest are complex, the chest CAD processing using the filtering processing described above cannot sufficiently remove the background image. Therefore, abnormal shadow detection performance is not improved thereby. For this reason, a method has been proposed for removing anatomical structures such as bones as a background image by generating an artificial image (see Japanese Unexamined Patent Publication No. 2005-020338, for example).

However, the conventional methods do not consider anatomical characteristics of ribs such as overlaps thereof. Therefore, accurate representation of texture of a subject has been difficult, which disrupts detection of an abnormal shadow in chest CAD processing.

### SUMMARY OF THE INVENTION

The present invention has been conceived based on consideration of the above circumstances. An object of the present invention is therefore to provide an image processing apparatus, an image processing method, and a program that enable accurate inference of a rib image based on a chest image.

A first image processing apparatus of the present invention comprises:

chest image storage means for storing chest images obtained by plain radiography of the chests of a plurality of subjects;

rib image generation means for generating rib images by extracting pixel value components contributing to ribs from values of pixels comprising the respective chest images;

rib overlap detection means for detecting rib overlaps where the ribs appear to overlap in rib regions in the respective rib images generated by the rib image generation means;

image normalization means for normalizing the rib images so as to cause positions of the rib overlaps detected by the rib overlap detection means in the respective chest images to agree in all the rib images;

rib image analysis means for carrying out analysis on pixel values of the rib images by applying a statistical method to the rib images having been normalized; and

rib image inference means for generating an inferred rib image by inferring pixel values of ribs in a chest image obtained by radiography of a predetermined subject, by using a result of the analysis by the rib image analysis means.

A first image processing method of the present invention comprises the steps of:

storing chest images obtained by plain radiography of the chests of a plurality of subjects in chest image storage means;

generating rib images by extracting pixel value components contributing to ribs from values of pixels comprising the respective chest images;

detecting rib overlaps where the ribs appear to overlap in rib regions in the respective rib images generated in the rib image generating step;

normalizing the rib images so as to cause positions of the rib overlaps detected in the rib overlap detecting step in the respective chest images to agree in all the rib images;

carrying out analysis on pixel values of the rib images by applying a statistical method to the rib images having been normalized; and

generating an inferred rib image by inferring pixel values of ribs in a chest image obtained by radiography of a predetermined subject, by using a result of the analysis.

A first program of the present invention causes a computer to function as:

chest image storage means for storing chest images obtained by plain radiography of the chests of a plurality of subjects;

rib image generation means for generating rib images by extracting pixel value components contributing to ribs from values of pixels comprising the respective chest images;

rib overlap detection means for detecting rib overlaps where the ribs appear to overlap in rib regions in the respective rib images generated by the rib image generation means;

image normalization means for normalizing the rib images so as to cause positions of the rib overlaps detected by the rib overlap detection means in the respective chest images to agree in all the rib images;

rib image analysis means for carrying out analysis on pixel values of the rib images by applying a statistical method to the rib images having been normalized; and

rib image inference means for generating an inferred rib image by inferring pixel values of ribs in a chest image obtained by radiography of a predetermined subject, by using a result of the analysis by the rib image analysis means.

The values of the pixels comprising the respective chest images are pixel values representing density corresponding to radiographed anatomical structures such as the ribs, a heart, and lung fields. At parts where soft tissues such as the heart and lung fields are in overlap with the ribs, the pixel values represent the density affected by density caused by the soft tissues and the ribs.

The pixel value components contributing to the ribs refer to pixel value components contributing to the ribs obtained by removing pixel value components affected by the anatomical structures other than the ribs from the pixel values comprising each of the chest images.

Normalization of the rib images refers to transformation of the ribs represented in the respective rib images so as to have a desired uniform shape.

The first image processing apparatus may further comprise image division means for dividing the ribs in the respective rib images into partial rib images individually representing the respective ribs. In this case, the image normalization means may normalize the partial rib images by transformation thereof so as to cause the positions of the rib overlaps in the partial rib images corresponding to each other to agree, after transforming the partial rib images into a predetermined normalized shape.

The predetermined normalized shape refers to a shape defined for use as a standard. Transforming the partial rib images into the predetermined normalized shape refers to transforming the partial rib images so as to have the uniform normalized shape.

The rib image analysis means may obtain principal component images by carrying out principal component analysis on the pixel values of the rib images of the respective subjects so that the rib image inference means can generate the inferred rib image by inferring the pixel values of the ribs of the predetermined subject through weighted addition of the principal component images.

The principal component images refer to images representing principal components obtained as a result of the principal component analysis on the pixel values in the rib images.

Furthermore, the rib image inference means may generate a rib image by extracting pixel value components contributing to the ribs from the pixel values comprising the chest image of the predetermined subject, for inferring pixel values of normal ribs of the subject from at least a part of the rib image.

A second image processing apparatus of the present invention comprises:

chest image storage means for storing a chest image obtained by plain radiography of the chest of a subject;

rib image generation means for generating a rib image by extracting pixel value components contributing to ribs from values of pixels comprising the chest image;

rib shape extraction means for extracting shapes of the respective ribs from the chest image or the rib image;

model rib shape setting means for setting a model rib shape corresponding to an anatomical rib structure along the shape of each of the ribs, based on the shape thereof among the shapes extracted by the rib shape extraction means and pixel values in a region thereof in the rib image; and

rib image inference means for generating an inferred rib image by inferring pixel values of the respective ribs in the chest image, based on the model rib shapes set by the model rib shape setting means.

A second image processing method of the present invention comprises the steps of:

storing a chest image obtained by plain radiography of the chest of a subject in chest image storage means;

generating a rib image by extracting pixel value components contributing to ribs from values of pixels comprising the chest image;

extracting shapes of the respective ribs from the chest image or the rib image;

setting a model rib shape corresponding to an anatomical rib structure along the shape of each of the ribs, based on the shape thereof among the shapes extracted by the shape extracting step and pixel values in a region thereof in the rib image; and

generating an inferred rib image by inferring pixel values of the respective ribs in the chest image, based on the model rib shapes set by the model rib shape setting step.

A second program of the present invention causes a computer to function as:

chest image storage means for storing a chest image obtained by plain radiography of the chest of a subject;

rib image generation means for generating a rib image by extracting pixel value components contributing to ribs from values of pixels comprising the chest image;

rib shape extraction means for extracting shapes of the respective ribs from the chest image or the rib image;

model rib shape setting means for setting a model rib shape corresponding to an anatomical rib structure along the shape of each of the ribs, based on the shape thereof among the shapes extracted by the rib shape extraction means and pixel values in a region thereof in the rib image; and

rib image inference means for generating an inferred rib image by inferring pixel values of the respective ribs in the chest image, based on the model rib shapes set by the model rib shape setting means.

The values of the pixels comprising the chest image are pixel values representing density corresponding to radiographed anatomical structures such as the ribs, a heart, and lung fields. At parts where soft tissues such as the heart and lung fields are in overlap with the ribs, the pixel values represent the density affected by density caused by the soft tissues and the ribs.

The pixel value components contributing to the ribs refer to pixel value components contributing to the ribs obtained by removing pixel value components affected by the anatomical structures other than the ribs from the pixel values comprising the chest image.

The model rib shapes refer to shapes corresponding to the anatomical rib structure, and enable calculation of the pixel values appearing in accordance with an amount of X rays passing through the ribs.

The model rib shapes are preferably tube-like shapes along long axes of the respective rib shapes.

A third image processing apparatus of the present invention comprises:

chest image storage means for storing a chest image obtained by plain radiography of the chest of a subject;

rib region inference means for inferring a rib region in the chest image;

non-rib region extraction means for extracting a non-rib region as a region other than the rib region from lung field regions in the chest image;

soft tissue image inference means for generating an inferred soft tissue image by inferring pixel value components contributing to soft tissues in the lung field regions in the chest image, based on an image of the non-rib region;

inferred bone image generation means for generating an inferred bone image comprising pixel value components contributing to ribs among pixel values of the chest image, through removal of the inferred soft tissue image from the chest image; and

rib region detection means for detecting a rib region in the inferred bone image.

A third image processing method of the present invention comprises the steps of:

storing a chest image obtained by plain radiography of the chest of a subject in chest image storage means;

inferring a rib region in the chest image;

extracting a non-rib region as a region other than the rib region from lung field regions in the chest image;

generating an inferred soft tissue image by inferring pixel value components contributing to soft tissues in the lung field regions in the chest image, based on an image of the non-rib region;

generating an inferred bone image comprising pixel value components contributing to ribs among pixel values of the chest image, through removal of the inferred soft tissue image from the chest image; and

detecting a rib region in the inferred bone image.

A third program of the present invention causes a computer to function as:

chest image storage means for storing a chest image obtained by plain radiography of the chest of a subject;

rib region inference means for inferring a rib region in the chest image;

non-rib region extraction means for extracting a non-rib region as a region other than the rib region from lung field regions in the chest image;

soft tissue image inference means for generating an inferred soft tissue image by inferring pixel value components contributing to soft tissues in the lung field regions in the chest image, based on an image of the non-rib region;

inferred bone image generation means for generating an inferred bone image comprising pixel value components contributing to ribs among pixel values of the chest image, through removal of the inferred soft tissue image from the chest image; and

rib region detection means for detecting a rib region in the inferred bone image.

The rib region refers to a region wherein the ribs are shown in the chest image. The non-rib region refers to a region excluding the rib region from the lung field regions in the chest image.

The values of the pixels comprising the chest image are pixel values representing density corresponding to radiographed anatomical structures such as the ribs, a heart, and the lung fields. At parts where the soft tissues such as the heart and the lung fields are in overlap with the ribs, the pixel values represent the density affected by density caused by the soft tissues and the ribs.

The pixel value components contributing to the soft tissues in the lung field regions in the chest image refer to pixel value components contributing to anatomical structures of the soft tissues obtained by removing an effect of the ribs from pixel values in the lung field regions in the chest image.

The pixel value components contributing to the ribs in the chest image refer to pixel value components contributing to the ribs obtained by removing an effect of anatomical structures other than the ribs from the pixel values comprising the chest image.

It is preferable for the soft tissue image inference means to generate the inferred soft tissue image based on a result of analysis of pixel values of soft tissues in chest images obtained by radiography of a large number of subjects, by use of statistical analysis means.

In the case where the analysis means is principal component analysis and obtains principal component images of the soft tissues as a result of the analysis, the soft tissue image inference means may generate the inferred soft tissue image by inferring pixel values of the soft tissues of the predetermined subject through weighted addition of the principal component images.

The principal component images refer to images representing principal components obtained as the result of the principal component analysis on the pixel values of the soft tissues.

According to the first image processing apparatus, the first image processing method, and the first program of the present invention, the rib images are generated by extraction of the pixel value components contributing to the ribs in the respective chest images, and the rib images are normalized so as to have the same positions of the rib overlaps in all the rib images. The normalized rib images are then analyzed by use of a statistical method, and the inferred rib image is generated by inferring normal ribs of the subject as an examination target from the chest image thereof, based on the result of the analysis. In this manner, density at the rib overlaps can be accurately represented. By removing the inferred rib image from the chest image, an image of soft tissues of the subject can be extracted accurately. Therefore, accuracy of detection of an abnormal shadow in lung fields can be improved.

In the case where the ribs in each of the rib images are separated into the partial rib images and subjected to transformation into the normalized shape, if the analysis is carried out by using the partial rib images normalized to have the same positions of the rib overlaps between the corresponding partial rib images, an effect caused by a difference in rib shapes among subjects can be weakened. In this manner, accuracy of the analysis is improved.

By inferring the rib image of the subject as the target of examination through the weighted addition of the principal component images obtained by principal component analysis on the pixel values of the rib images, the image of the normal ribs of the subject represented in the rib image can be inferred as a combination of a small number of the principal component images.

By generating the rib images from the chest images and by using the result of principal component analysis carried out on the pixel values of the rib images, the pixel values of the normal ribs of the subject can be accurately inferred.

According to the second image processing apparatus, the second image processing method, and the second program of the present invention, the rib shapes are extracted from the chest image or the rib image, and the model rib shape is set along each of the rib shapes. The pixel values of the ribs in the chest image are then inferred. In this manner, density corresponding to the anatomical rib structure can be inferred accurately. In addition, by removing the inferred rib image generated in this manner from the chest image, a soft tissue image of the subject as a target of examination can be extracted accurately. In this manner, accuracy of abnormal shadow detection in lung fields can be improved.

By using the tube-like shape along the long axis of each of the ribs as the model rib shape, a result can be obtained in agreement with an anatomical characteristic of ribs.

According to the third image processing apparatus, the third image processing method, and the third program of the present invention, the soft tissue image is inferred from the non-rib region as the region excluding the rib region from the chest image, and the inferred soft tissue image is removed from the chest image for generating the inferred bone image. By detecting the rib region in the inferred bone image not affected by the soft tissues, the rib region can be detected with accuracy.

Furthermore, by inferring the soft tissue image based on principal component analysis from the non-rib region excluding the rib region, the soft tissue image radiographed in overlap with the ribs can be inferred. Therefore, the rib region can be inferred accurately in the bone image not affected by the soft tissues.

### BRIEF DESCRIPTION OF THE DRAWINGS

FIG. 1 shows the configuration of a first image processing apparatus of the present invention;

FIGS. 2A and 2B show an example of a result of principal component analysis carried out on soft tissue images;

FIGS. 3A and 3B show another example of a result of principal component analysis carried out on the soft tissue images;

FIG. 4 shows rib image normalization;

FIG. 5 shows rib overlaps;

FIG. 6 shows an example of a normalized rib image;

FIGS. 7A and 7B show an example of a result of principal component analysis carried out on rib images;

FIGS. 8A and 8B show another example of a result of principal component analysis carried out on the rib images;

FIG. 9 is a flow chart showing procedures carried out in the first image processing apparatus;

FIG. 10 shows the configuration of a second image processing apparatus of the present invention;

FIGS. 11A and 11B show an example of a result of principal component analysis carried out on soft tissue images;

FIGS. 12A and 12B show another example of a result of principal component analysis carried out on the soft tissue images;

FIG. 13 shows extracted rib shapes;

FIGS. 14A and 14B show distributions of pixel values of a rib;

FIG. 15 shows an example of a model rib shape;

FIG. 16 is a flow chart showing procedures carried out in the second image processing apparatus;

FIG. 17 shows the configuration of a third image processing apparatus of the present invention;

FIGS. 18A and 18B show an example of a result of principal component analysis carried out on soft tissue images;

FIGS. 19A and 19B show another example of a result of principal component analysis carried out on the soft tissue images;

FIGS. 20A and 20B show examples of a chest image and a non-rib region;

FIG. 21 shows an example of an inferred soft tissue image;

FIG. 22 shows an example of an inferred bone image;

FIGS. 23A to 23C show processes of principal component analysis on rib shapes; and

FIG. 24 is a flow chart showing procedures carried out in the third image processing apparatus.

### DESCRIPTION OF THE PREFERRED EMBODIMENTS

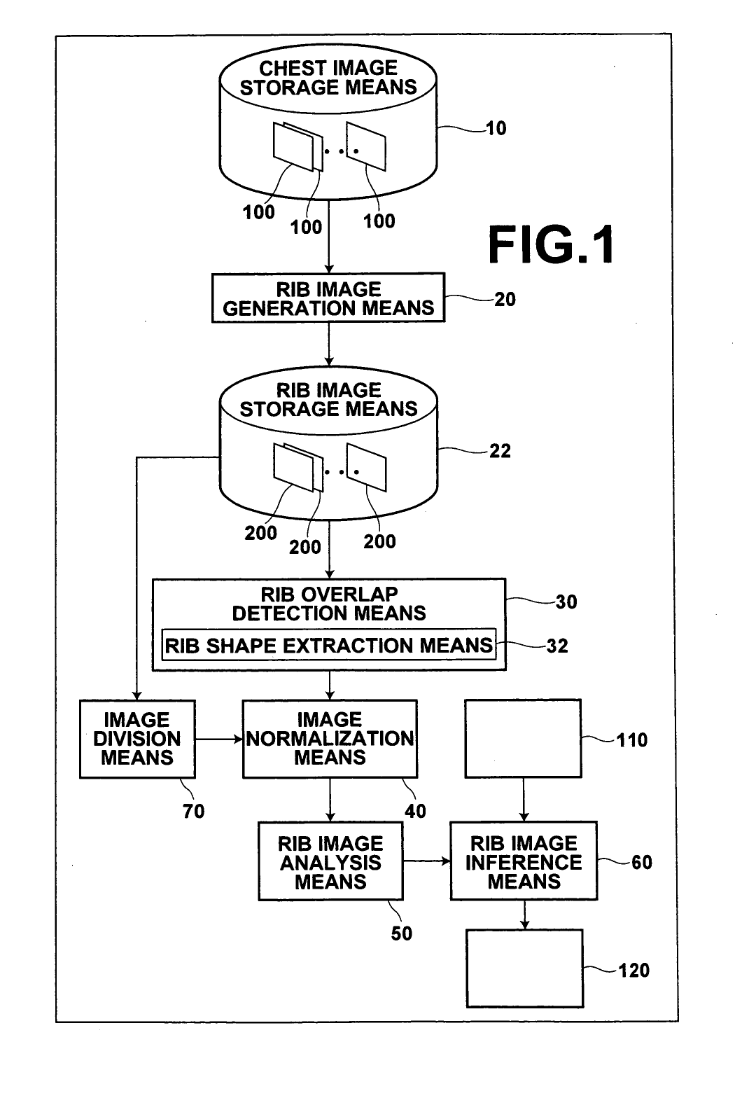

Hereinafter, a first embodiment of the present invention is described with reference to the accompanying drawings. FIG. 1 shows the configuration of an image processing apparatus of the first embodiment.

As shown in FIG. 1, an image processing apparatus 1 comprises chest image storage means 10, rib image generation means 20, rib image storage means 22, rib overlap detection means 30, image normalization means 40, rib image analysis means 50, and rib image inference means 60. The chest image storage means 10 stores a plurality of chest images 100 obtained by plain radiography of the chests of subjects. The rib image generation means 20 generates rib images 200 by extraction of pixel value components contributing to ribs from values of pixels comprising the respective chest images 100. The rib image storage means 22 stores the rib images 200. The rib overlap detection means 30 detects rib overlaps at which ribs appear to overlap in rib regions in the respective rib images 200. The image normalization means 40 normalizes the rib images 200 so as to cause positions of the rib overlaps detected by the rib overlap detection means 30 to agree among all the rib images 200. The rib image analysis means 50 analyzes the pixel values of the rib images by applying a statistical method to the normalized rib images. The rib image inference means 60 generates an inferred rib image 120 by inferring pixel values of ribs in a chest image 110 obtained by radiography of a predetermined subject.

The rib overlap detection means 30 has rib shape extraction means 32. The rib overlap detection means 30 detects the rib overlaps in the rib regions in extracted rib shapes.

The image processing apparatus 1 also comprises image division means 70 for dividing ribs in each of the rib images into partial rib images by separating the ribs into individual ribs. The image normalization means 40 transforms the respective partial rib images into a predetermined normalized shape, and normalizes the partial rib images so as to cause the positions of the rib overlaps to agree between the partial rib images corresponding to each other.

The chest images 100 (110) are obtained by plain radiography of subjects by use of a CR (Computed Radiography) apparatus or the like. In the images obtained by plain radiography, each anatomical structure in the chest of each of the subjects appears as pixel values of density corresponding to X-ray transmittance (or absorption rate) thereof. Since ribs, for example, have a high X-ray absorption rate, the ribs look white in the chest images 100 (110). However, since the density in the chest images is affected by the transmittance of all organs through which X-ray passes, the ribs appear in density affected by other organs such as a heart or lung fields overlapping the ribs. For this reason, even in the case where the ribs have the same thickness, the ribs appear in different density in each of the chest images 100, depending on an organ such as the heart or the lung fields under the ribs.

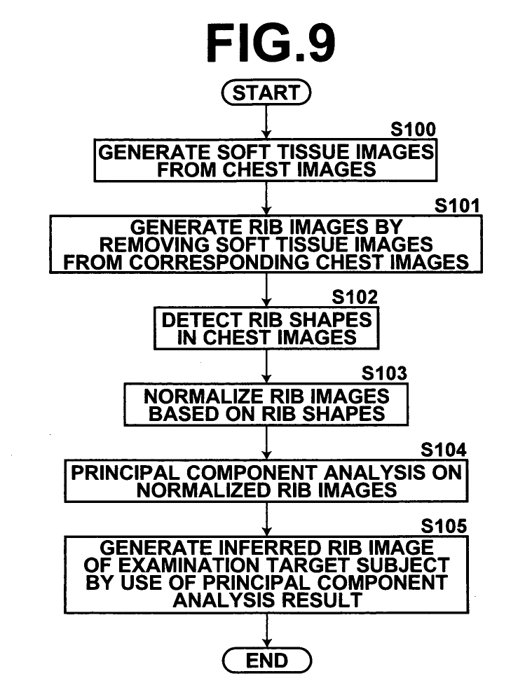

Following a flow chart in FIG. 9 is described a flow of procedures in the image processing apparatus 1 for inferring a rib image that is free from the effect of organs under the ribs in the chest image of the subject as a target of examination.

(1) Generation of Rib Images

The rib image generation means 20 generates the rib images by extracting the pixel value components contributing to the ribs from the pixel values of the chest images 100 stored in the chest image storage means 10. More specifically, the rib images not affected by soft tissues are generated by removing soft tissue images from the chest images 100.

The soft tissue images are generated artificially by using a result of analysis of a plurality of soft tissue images obtained by energy subtraction processing in soft tissue image analysis processing (S100). In the soft tissue image analysis processing, principal component analysis is carried out on the plurality of soft tissue images obtained by energy subtraction processing, for finding principal components (vector components) of the soft tissue images. The principal components are linearly independent, and the soft tissue images can be reproduced artificially by use of a small number of the linearly independent vector components.

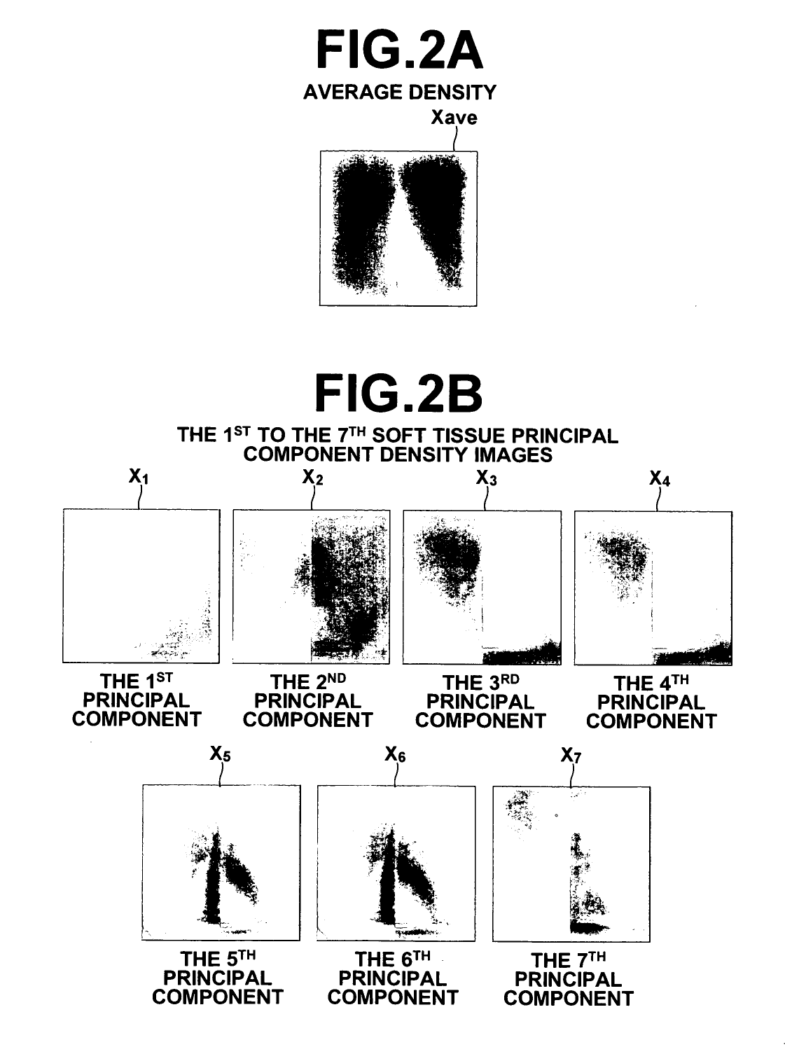

Firstly, each of the soft tissue images is transformed into a normalized shape such as a rectangle shown in FIGS. 2A and 2B.

Let coordinates before the transformation and corresponding coordinates after the transformation be B(x_{B}, y_{B}) and A(x_{A}, y_{A}), respectively. At the time of the transformation, the y-coordinate of B is converted into the corresponding y-coordinate of A according to Equation (1) below if the y-coordinate of the highest point and the lowest point in lung fields are represented by y_{B,up} and y_{B,down} for B and by y_{A,up} and y_{A,down} for A, respectively:

$${y}_{A} = {y}_{A , up} + \frac{{y}_{A , down} - {y}_{A , up}}{{y}_{B , down} - {y}_{B , up}} ( {y}_{B} - {y}_{B , up} ) \qquad ( 1 )$$

Let positions of the right and left lung fields at y_{B} be represented by x_{B,left} and x_{B,right} while positions thereof at y_{A} be denoted by x_{A,left} and x_{A,right}. The transformation is carried out so as to cause the positions of the right and left lung fields at y_{B} to agree with the positions thereof at y_{A}according to Equation (2) below:

$${x}_{A} = {x}_{A , left} + \frac{{x}_{A , right} - {x}_{A , left}}{{x}_{B , right} - {x}_{B , left}} ( {x}_{B} - {x}_{B , left} ) \qquad ( 2 )$$

An average image Xₐᵥₑ (shown by FIG. 2A) of the soft tissue images having been transformed into the rectangular shape (hereinafter referred to as the average soft tissue density image) is found together with principal component soft tissue density images Xᵢ (i=1, 2, 3, . . . ) as the first to the n^{th} principal components (see FIG. 2B wherein the first to the seventh principal components are shown) obtained by principal component analysis on subtraction images between the soft tissue images and the average soft tissue density image. A soft tissue image X can then be represented by Equation (3) below, by the average soft tissue density image Xₐᵥₑ and weighted addition of the principal component soft tissue density images Xᵢ:
X=Xₐᵥₑ+Σᵢaᵢ·Xᵢ (3)
where
X is a vector whose components are pixel values in the soft tissue image,
Xₐᵥₑ is a vector whose components are pixel values in the average soft tissue density image,
Xᵢ is a principal component vector representing the i^{th} principal component soft tissue density image, and
aᵢ is a weight coefficient for the i^{th} principal component vector.

For inferring the soft tissue images of the chest images 100 of the respective subjects, the weight coefficients are determined based on the respective chest images 100 of the subjects so as to cause the values of X to approximate the pixel values of the soft tissues other than the ribs according to Equation (3).

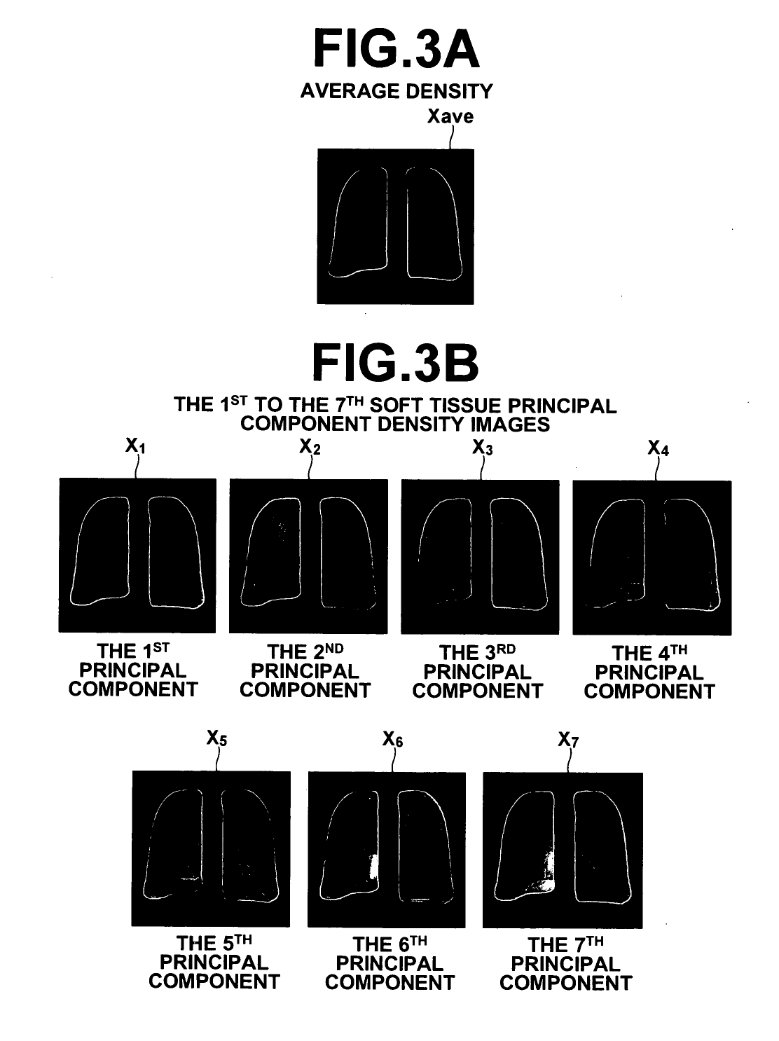

Alternatively, the soft tissue images may be normalized into an average shape of the soft tissues. In this case are generated an average soft tissue density image shown by FIG. 3A and principal component density images shown by FIG. 3B. Weight coefficients are then determined based on the chest images 100 of the subjects so as to cause values calculated by use of the weight coefficients to agree with density of the soft tissues other than the ribs, for inferring the soft tissue images X of the respective subjects.

By subtracting the inferred soft tissue images X from the corresponding chest images 100, the rib images 200 are generated, excluding density contributing to the soft tissues in the chest images 100. The rib images 200 are stored in the rib image storage means 22 (S101).

(2) Detection of Rib Shapes

The rib shape extraction means 32 detects the rib shapes in the respective chest images 100 (110) (S102). More specifically, an edge image is generated from each of the chest images 100 by use of an edge extraction filter, and parabolic lines similar to ribs are found in the edge image by use of Huff transform or the like that detects parabolic lines (see Peter de Souza, “Automatic Rib Detection in Chest Radiographs”, Computer Vision, Graphics, and Image Processing, Vol. 23, Issue 2, pp. 129-161, 1983, for example).

(3) Normalization of Rib Images

The rib overlaps at which the ribs appear to overlap in each of the chest images look more whitish than other parts of the ribs without the overlaps, and characteristics different from the parts without the overlaps are also shown. Furthermore, the rib overlaps on the third rib with other ribs, for example, appear at substantially the same position even among different subjects. Therefore, when the pixel values of the ribs of the plurality of subjects are analyzed, the analysis can be carried out with high accuracy by normalization of the shapes so as to cause positions of the same characteristics to appear at the same positions.

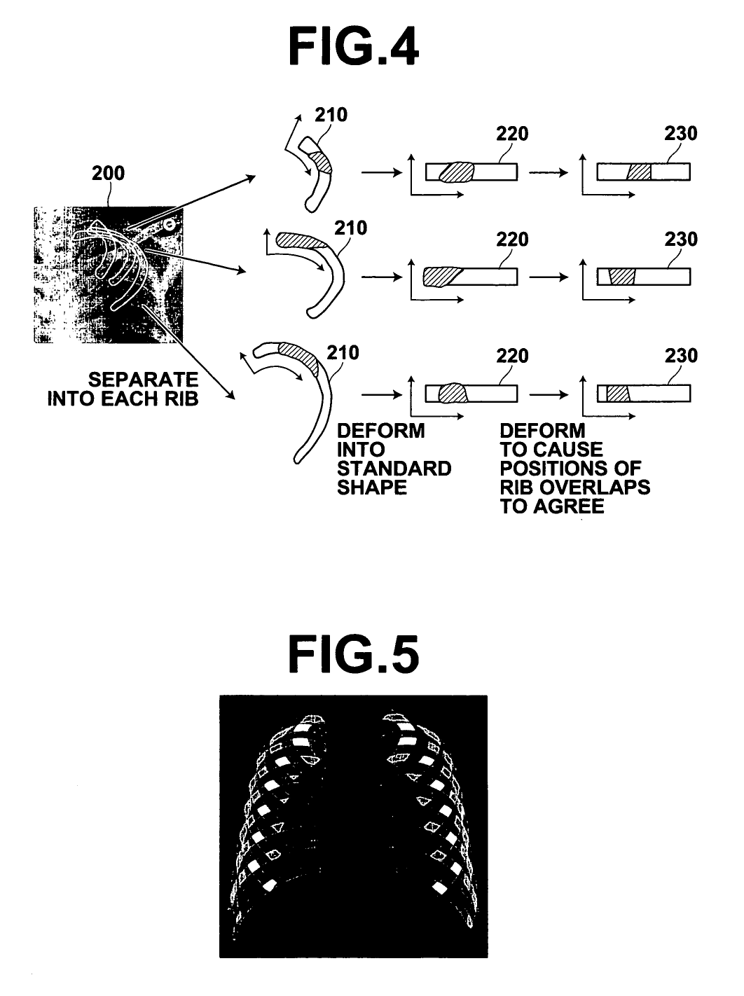

For this reason, the rib overlap detection means 30 recognizes the rib region in which the ribs appear, by superposing the rib shapes detected by the rib shape extraction means 32 onto each of the rib images 200 (see the left image in FIG. 4), and detects the rib overlaps at which the ribs appear to overlap in the rib region.

Thereafter, the image division means 70 separates the ribs in the corresponding rib images 200 into the individual ribs as shown in FIG. 4, based on the rib shapes detected in the processes of (2), for generating partial rib images 210.

The image normalization means 40 then transforms the shapes of the partial rib images 210 into a normalized shape 220 such as a rectangle shown in FIG. 4. The image normalization means 40 then transforms the shape of the rectangular partial rib images into a shape 230 by scaling so as to cause the rib overlaps to be positioned at the same positions. Since the respective ribs have different positions of the rib overlaps depending on the ordinal number thereof (that is, where the ribs are located), the shape is transformed so as to cause the positions of the rib overlaps to agree among the ribs of the same part.

However, depending on the rib shapes of the respective subjects and on a direction of radiography, how the ribs overlap slightly vary. For example, for the third rib, one of the subjects may have 3 rib overlaps with other ribs while another one of the subjects may have 2 rib overlaps. Therefore, as shown in FIG. 5, the partial rib images may be normalized by scaling so as to cause main rib overlaps (the portions represented in white where ribs necessarily overlap among a large number of subjects) to be positioned at the same positions.

The partial rib images corresponding to the 10 ribs each in the right and the left of each of the subjects are transformed to have the rectangular shape as has been described above, and the partial rib images normalized to have substantially the same positions of the rib overlaps are unified to form a normalized rib image 240 shown in FIG. 6 (S103).

(4) Analysis of Rib Images

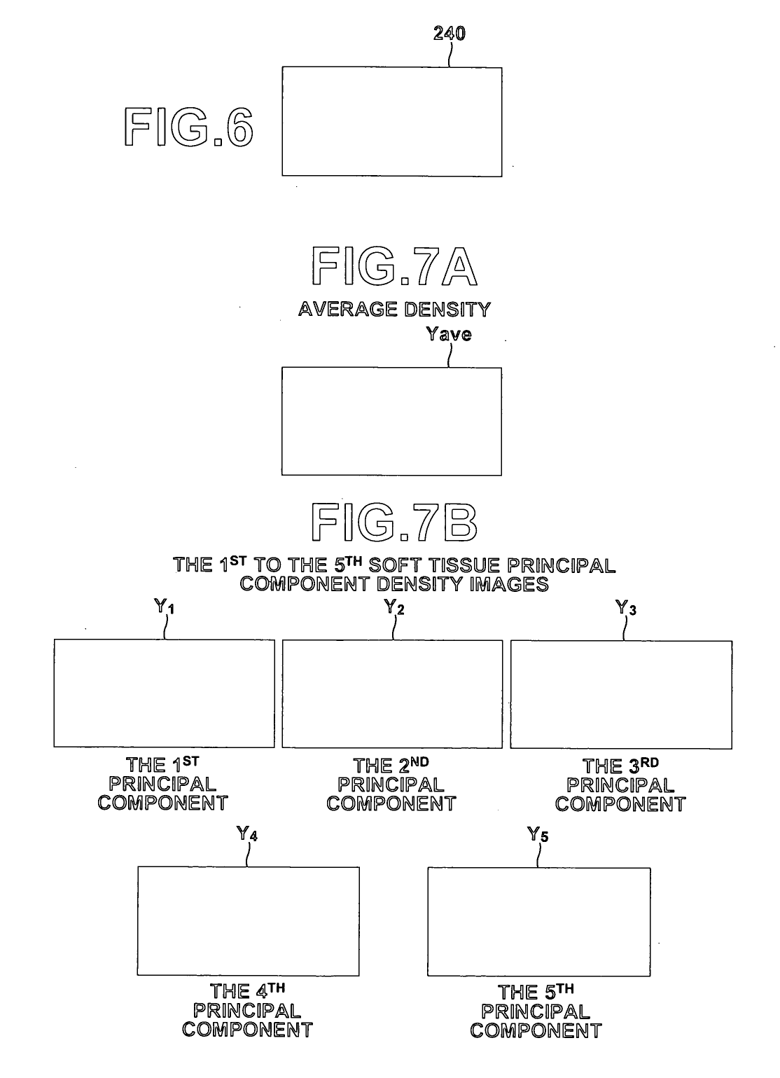

The rib image analysis means 50 carries out principal component analysis on the normalized rib images 240 generated by normalization of the chest images as has been described above. The rib image analysis means 50 generates an average rib density image Yₐᵥₑ shown in FIG. 7A from the normalized rib images 240, and carries out principal component analysis on subtraction images between the normalized rib images 240 and the average rib density image Yₐᵥₑ (S104) As a result, the first to the n^{th} principal component rib density images Yᵢ (i=1, 2, 3, . . . n) shown in Figure 7B are obtained, for example. The respective rib images 200 are represented according to Equation (4) below, by the average rib density image Yₐᵥₑ and weighted addition of the principal component rib density images Yi as images of the first to the n^{th} (n=5 in FIG. 7B) principal components (the principal component images) obtained by the principal component analysis:
Y=Yₐᵥₑ+bᵢ·Yᵢ (4)
where
Y is a vector whose components are pixel values of a normalized rib image,
Yₐᵥₑ is a vector whose components are pixel values in the average rib density image,
Yᵢ is a principal component vector representing the i^{th} principal component rib density image, and
bᵢ is a weight coefficient for the i^{th} principal component vector.

(5) Inference of Rib Image

The rib image inference means 60 determines the weight coefficients bᵢ in Equation (4) so as to cause the values calculated by use of the weight coefficients to agree with the density of the ribs of the subject to be examined, for inferring the pixel values of the rib image of the subject.

Firstly, the rib image is extracted from the chest image 110 of the subject according to the processes described in (1) above, and rib shapes are extracted according to the processes of (2). A normalized rib image is generated through normalization of the rib image of the subject according to the processes described in (3) above, and the weight coefficients bᵢ in Equation (4) are determined so as cause the pixel values calculated by use of the weight coefficients to agree with the pixel values of the normalized rib image of the subject. In this manner, the pixel values of the normalized rib image are inferred. At this time, the pixel values of the whole rib image can be inferred by finding the weight coefficients bᵢ to cause the values to agree with the pixel values of a part of the normalized rib image of the subject. Furthermore, the rib image obtained in this manner is transformed so as to agree with the rib shapes of the subject, for generating an inferred rib image 120 (S105).

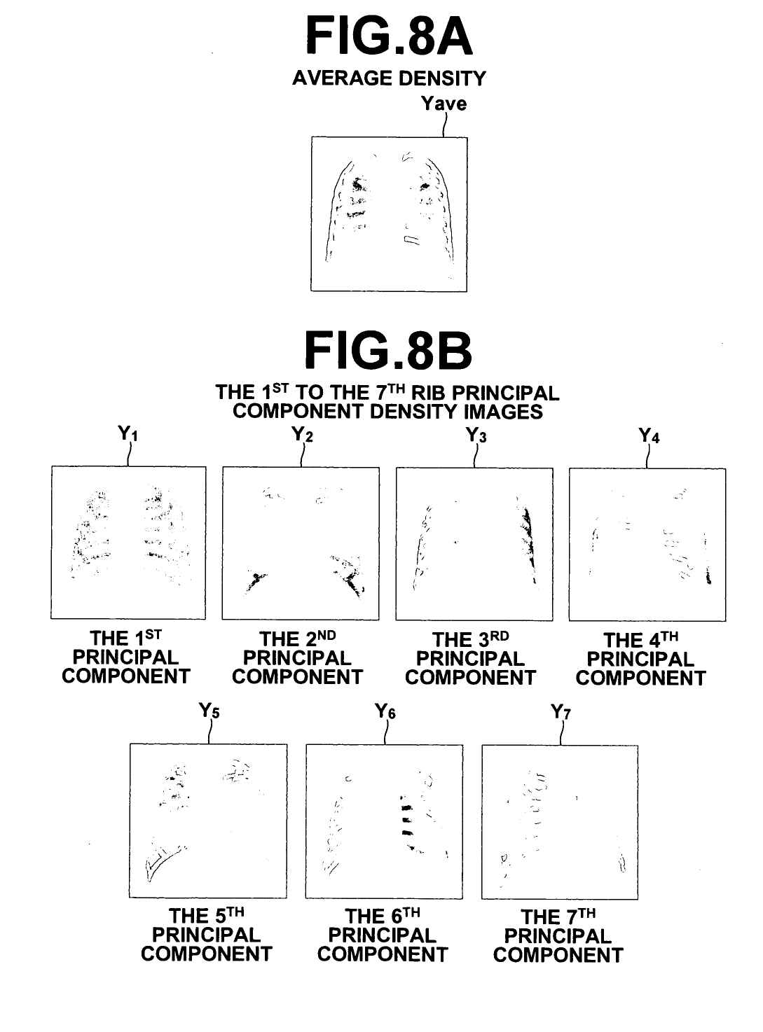

In the above description, when the rib images are normalized, each of the ribs is transformed into the rectangular shape. However, the ribs may be transformed to have normalized shapes shown in FIG. 8 so that the principal component images shown in FIG. 8B can be obtained through the principal component analysis thereon.

As has been described above, according to this method, the pixel values of the ribs can be inferred with accuracy. By using the rib image obtained in this manner, the ribs are removed from the original image. In this manner, a soft tissue image can be extracted accurately, which enables accurate detection of an abnormal shadow caused by cancer or the like.

By installing a program comprising the means described above in a computer, the computer can function as the image processing apparatus.

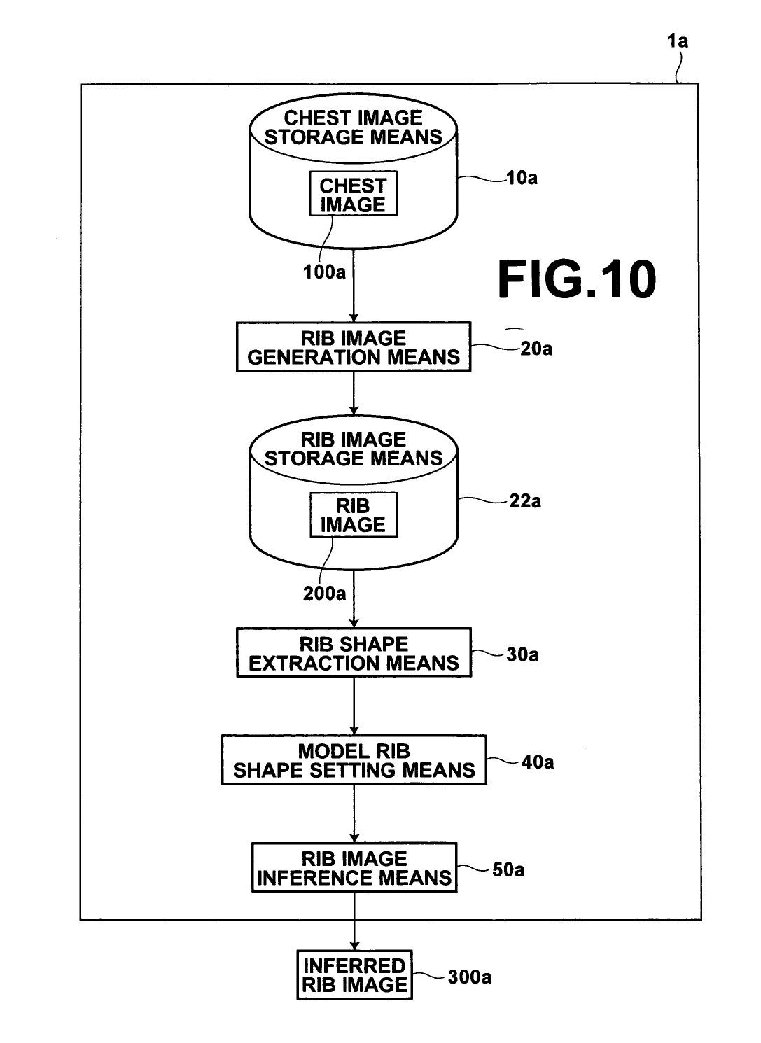

A second embodiment of the present invention is described next with reference to the accompanying drawings. FIG. 10 shows the configuration of an image processing apparatus in this embodiment.

As shown in FIG. 10, an image processing apparatus 1a comprises chest image storage means 10a, rib image generation means 20a, rib image storage means 22a, rib shape extraction means 30a, model rib shape setting means 40a, and rib image inference means 50a. The chest image storage means 10a stores a chest image 100a obtained by plain radiography of the chest of a subject. The rib image generation means 20a generates a rib image 200a by extracting pixel value components contributing to ribs from values of pixels comprising the chest image 100a, and stores the rib image 200a in the rib image storage means 22a. The rib shape extraction means 30a extracts shapes of the ribs from the chest image 100a or the rib image 200a. The model rib shape setting means 40a sets a model rib shape corresponding to an anatomical rib structure along the shape of each of the ribs, according to the shape thereof and pixel values thereof in the rib image. The rib image inference means 50a generates an inferred rib image 300a by inferring the pixel values of the ribs in the chest image 100a based on the model rib shapes.

The chest image 100a is obtained by plain radiography of the subject by use of a CR (Computed Radiography) apparatus or the like. In the image obtained by plain radiography, each anatomical structure in the chest of the subject appears as pixel values of density corresponding to X-ray transmittance (or absorption rate) thereof. Since ribs, for example, have a high X-ray absorption rate, the ribs look white in the chest image 100a. However, since the density in the chest image is affected by the transmittance of all organs through which X-ray passes, the ribs appear in density affected by other organs such as a heart or lung fields overlapping the ribs. For this reason, even in the case where the ribs have the same thickness, the ribs appear in different density in the chest image 100a, depending on an organ such as the heart or the lung fields under the ribs.

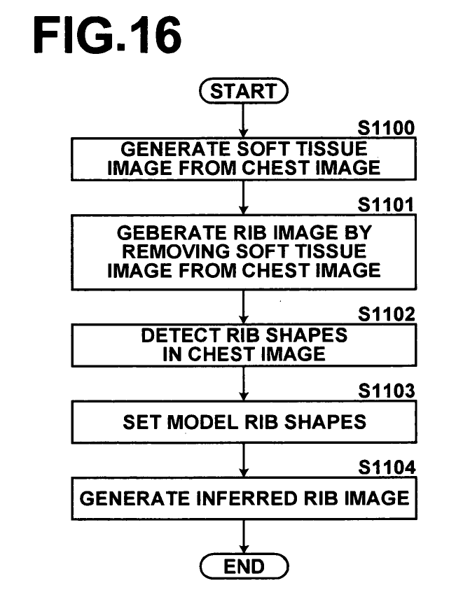

Following a flow chart in FIG. 16 is described a flow of procedures in the image processing apparatus 1a for inferring the rib image that is free from the effect of organs under the ribs in the chest image of the subject as a target of examination.

The rib image generation means 20a generates the rib image by extracting the pixel value components contributing to the ribs from the pixel values of the chest image 100a stored in the chest image storage means 10a. More specifically, the rib image not affected by soft tissues is generated by removing a soft tissue image from the chest image 100.

The soft tissue image is generated artificially by using a result of analysis of a plurality of soft tissue images obtained by energy subtraction processing in soft tissue image analysis processing. In the soft tissue image analysis processing, principal component analysis is carried out on the plurality of soft tissue images obtained by energy subtraction processing, for finding principal components (vector components) of the soft tissue images. The principal components are linearly independent, and the soft tissue images can be reproduced artificially by use of a small number of the linearly independent vector components.

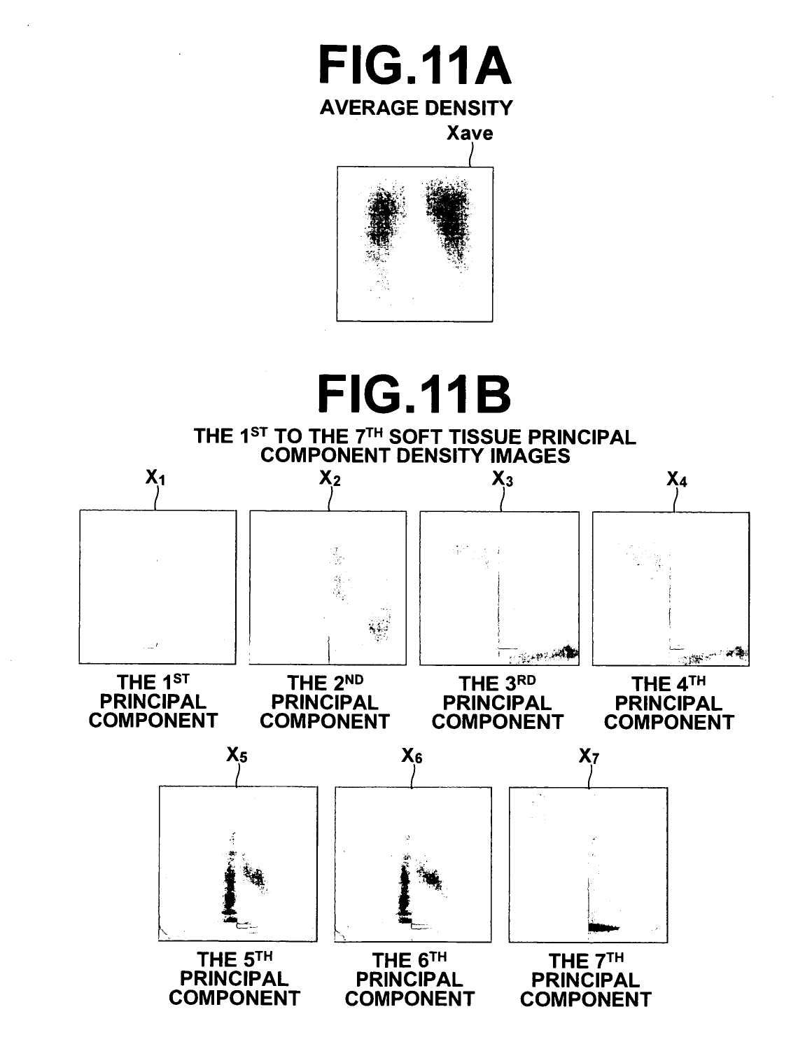

Firstly, each of the soft tissue images is transformed into a normalized shape such as a rectangle shown in FIGS. 11A and 11B.

Let coordinates before the transformation and corresponding coordinates after the transformation be B(x_{B}, y_{B}) and A(x_{A}, y_{A}), respectively. At the time of the transformation, the y-coordinate of B is converted into the corresponding y-coordinate of A according to Equation (1) below if the y-coordinate of the highest point and the lowest point in lung fields are represented by y_{B,up} and y_{B,down} for B and by y_{A,up} and y_{A,down} for A, respectively:

$${y}_{A} = {y}_{A , up} + \frac{{y}_{A , down} - {y}_{A , up}}{{y}_{B , down} - {y}_{B , up}} ( {y}_{B} - {y}_{B , up} ) \qquad ( 1 )$$

Let positions of the right and left lung fields at y_{B} be represented by x_{B,left} and x_{B,right} while positions thereof at y_{A}be denoted by x_{A,left} and x_{A,right}. The transformation is carried out so as to cause the positions of the right and left lung fields at y_{B} to agree with the positions thereof at y_{A} according to Equation (2) below:

$${x}_{A} = {x}_{A , left} + \frac{{x}_{A , right} - {x}_{A , left}}{{x}_{B , right} - {x}_{B , left}} ( {x}_{B} - {x}_{B , left} ) \qquad ( 2 )$$

An average image Xₐᵥₑ (shown by FIG. 11A) of the soft tissue images having been transformed into the rectangular shape (hereinafter referred to as the average soft tissue density image) is found together with principal component soft tissue density images Xᵢ (i=1, 2, 3, . . . ) as the first to the n^{th} principal components (see FIG. 11B wherein the first to the seventh principal components are shown) obtained by principal component analysis on subtraction images between the soft tissue images and the average soft tissue density image. A soft tissue image X can then be represented by Equation (3) below, by the average soft tissue density image Xₐᵥₑ and weighted addition of the principal component soft tissue density images Xᵢ:
X=Xₐᵥₑ+Σᵢaᵢ·Xᵢ (3)
where
X is a vector whose components are pixel values in the soft tissue image,
Xₐᵥₑ is a vector whose components are pixel values in the average soft tissue density image,
Xᵢ is a principal component vector representing the i^{th} principal component soft tissue density image, and
aᵢ is a weight coefficient for the i^{th} principal component vector.

For inferring the soft tissue image of the chest image 100a of the subject, the weight coefficients are determined based on the chest image 100a of the subject so as to cause the values of X to agree with the pixel values of soft tissues other than the ribs according to Equation (3).

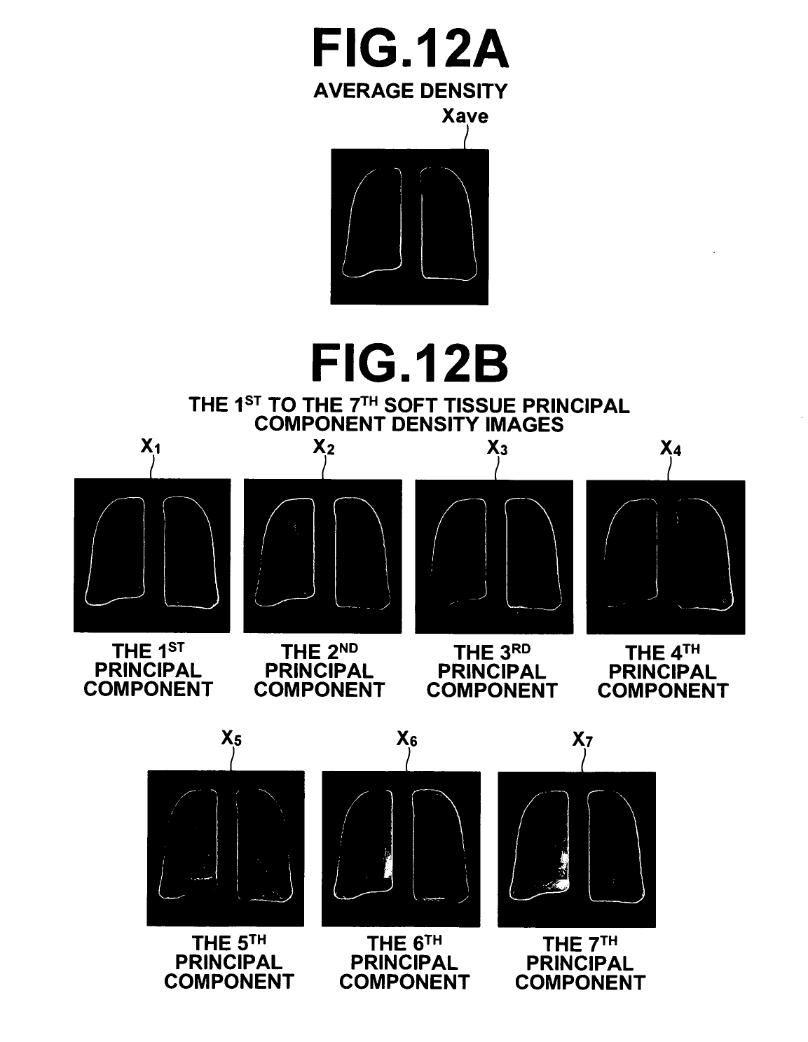

Alternatively, the soft tissue images may be normalized into an average shape of soft tissues. In this case are generated an average soft tissue density image shown by FIG. 12A and principal component density images shown by FIG. 12B. Weight coefficients are then determined based on the chest image 100a of the subject so as to cause values calculated by use of the weight coefficients to agree with density of the soft tissues other than the ribs, for inferring the soft tissue image X of the subject.

By subtracting the inferred soft tissue image X from the chest image 100a, the rib image 200a as extraction of the pixel value components contributing to the ribs is generated, excluding density contributing to the soft tissues in the chest images 100. The rib image 200a is stored in the rib image storage means 22a (S1101).

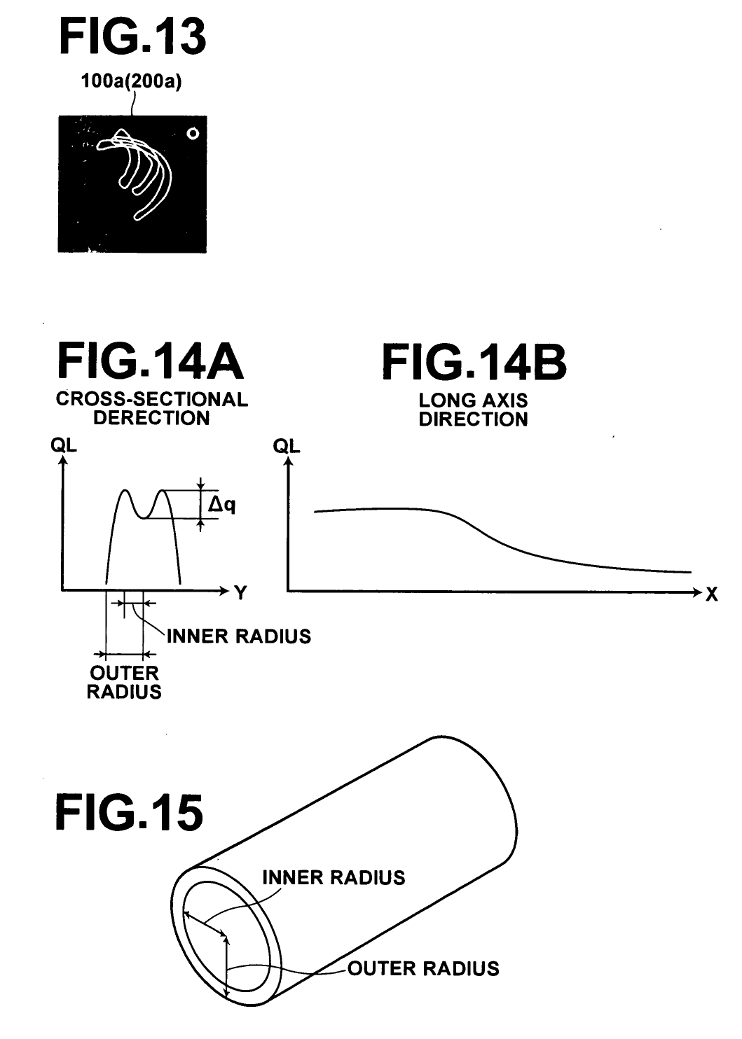

The rib shape extraction means 30a detects the rib shapes in the chest image 100a (S1102). More specifically, an edge image is generated from the chest image 100a by use of an edge extraction filter, and parabolic lines similar to ribs are found in the edge image by use of Huff transform or the like that detects parabolic lines (see Peter de Souza, “Automatic Rib Detection in Chest Radiographs”, Computer Vision, Graphics, and Image Processing, Vol. 23, Issue 2, pp. 129-161, 1983, for example). In this manner, the rib shapes can be detected as shown in FIG. 13.

The case where the rib shapes are extracted from the chest image 100a has been described above. However, the rib shapes may be extracted from the rib image 200a in the same manner.

Since bone tissues of the outer side of ribs have low X-ray transmittance, the ribs look white in the rib image 200a with high QL values. On the contrary, tissues of the inner side of ribs have slightly higher X-ray transmittance, and the QL values become lower at the inside of the ribs. In other words, the QL values become smaller near a center axis of each rib than a periphery thereof. Therefore, the QL value along a direction Y that crosses a centerline of each rib becomes smaller by Δq at the center than the outer side thereof, as shown in FIG. 14A. In addition, each rib starting from the base thereof becomes gradually thinner along a long axis thereof. Therefore, the QL value becomes smaller along a direction X of the long axis thereof, as shown in FIG. 14B. A function f(X) of the QL value can be represented by a three-dimensional polynomial, for example.

For this reason, the model rib shape setting means 40a assumes a model rib shape having the high QL values at the periphery thereof but the low QL values at the center axis, according to the anatomical rib structure. The model rib shape setting means 40a therefore sets the model rib shape along each of the ribs extracted by the rib shape extraction means 30a (S1103).

For example, a tube-like shape shown in FIG. 15 is assumed so as to correspond to the anatomical structure of the ribs. The tube-like shape is set along the long axis of each of the ribs shown in FIG. 13. The inner and outer radii and thickness of the tube in the model shape are determined so as to cause pixel values of the tube shape projected onto a two-dimensional plane to become closer to the pixel values in the rib image.

The rib image inference means 50a then infers the pixel values of the ribs based on the pixel values of the model shape projected onto the two-dimensional plane, for generating the inferred rib image 300a (S1104).

As has been described above in detail, according to this method, the pixel values of the ribs can be inferred accurately according to the anatomical structure thereof. If the ribs are removed from the original image by using the rib image obtained in this manner, the soft tissue image can be extracted accurately. Therefore, an abnormal shadow caused by cancer or the like can be detected accurately therein.

By installing a program having the means described above in a computer, the computer can function as the image processing apparatus.

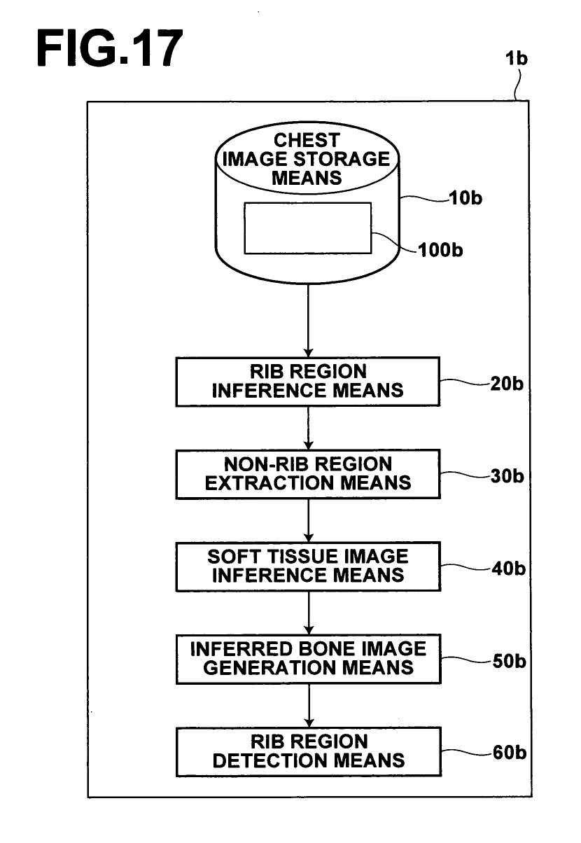

A third embodiment of the present invention is described next with reference to the accompanying drawings. FIG. 17 shows the configuration of an image processing apparatus in the third embodiment.

As shown in FIG. 17, an image processing apparatus 1b comprises chest image storage means 10b, rib region inference means 20b, non-rib region extraction means 30b, soft tissue image inference means 40b, inferred bone image generation means 50b, and rib region detection means 60b. The chest image storage means 10a stores a chest image 100b obtained by plain radiography of the chest of a subject. The rib region inference means 20b infers a rib region in the chest image. The non-rib region extraction means 30b extracts a non-rib region excluding the rib region from lung field regions in the chest image. The soft tissue image inference means 40b generates an inferred soft tissue image by inferring pixel value components contributing to soft tissues in the lung field regions in the chest image. The inferred bone image generation means 50b generates an inferred bone image comprising pixel value components contributing to ribs in pixel values in the chest image, through removal of the inferred soft tissue image from the chest image. The rib region detection means 60b detects a rib region in the inferred bone image.

The chest image 100b is obtained by plain radiography of the subject by use of a CR (Computed Radiography) apparatus or the like. In the image obtained by plain radiography, each anatomical structure in the chest of the subject appears as pixel values of density corresponding to X-ray transmittance (or absorption rate) thereof. Since ribs, for example, have a high X-ray absorption rate, the ribs look white in the chest image 100b. However, since the density in the chest image is affected by the transmittance of all organs through which X-ray passes, the ribs appear in density affected by other organs such as a heart or lung fields overlapping the ribs. For this reason, even in the case where the ribs have the same thickness, the ribs appear in different density in the chest image 100b, depending on an organ such as the heart or the lung fields under the ribs.

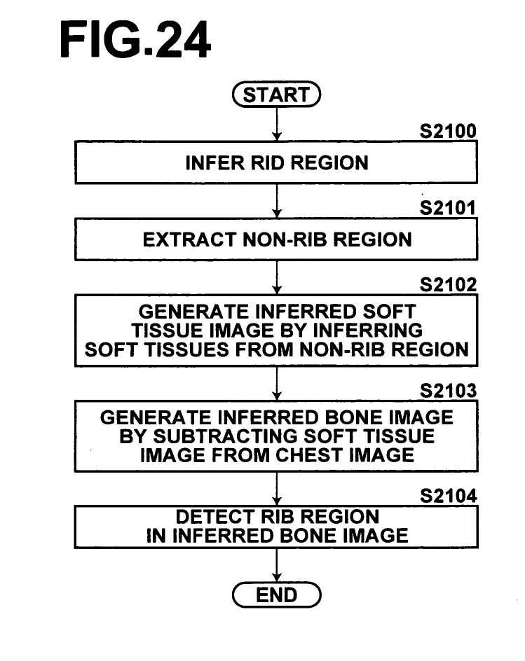

Following a flow chart in FIG. 24 is described a flow of procedures in the image processing apparatus 1b for detecting the rib region by removing the effect of organs under the ribs in the chest image of the subject as a target of examination.

The rib region inference means 20b recognizes rib shapes in the chest image 100b stored in the chest image storage means 10b. More specifically, an edge image is generated from the chest image 100b by use of an edge extraction filter, and parabolic lines similar to ribs are found in the edge image by use of Huff transform or the like that detects parabolic lines (see Peter de Souza, “Automatic Rib Detection in Chest Radiographs”, Computer Vision, Graphics, and Image Processing, Vol. 23, Issue 2, pp. 129-161, 1983, for example). In this manner, the rib shapes are detected. The rib region is then inferred from the rib shapes (S2100). The rib region inferred from the chest image 100b in this manner has low accuracy, due to the effects caused by the soft tissue structures such as the heart and blood vessels in the chest image 100b.

Thereafter, the inferred soft tissue image is generated from the non-rib region as a region excluding the rib region from the lung field regions in the chest image 100b, and detects the accurate rib region in the inferred bone image generated by excluding the inferred soft tissue image from the chest image 100b.

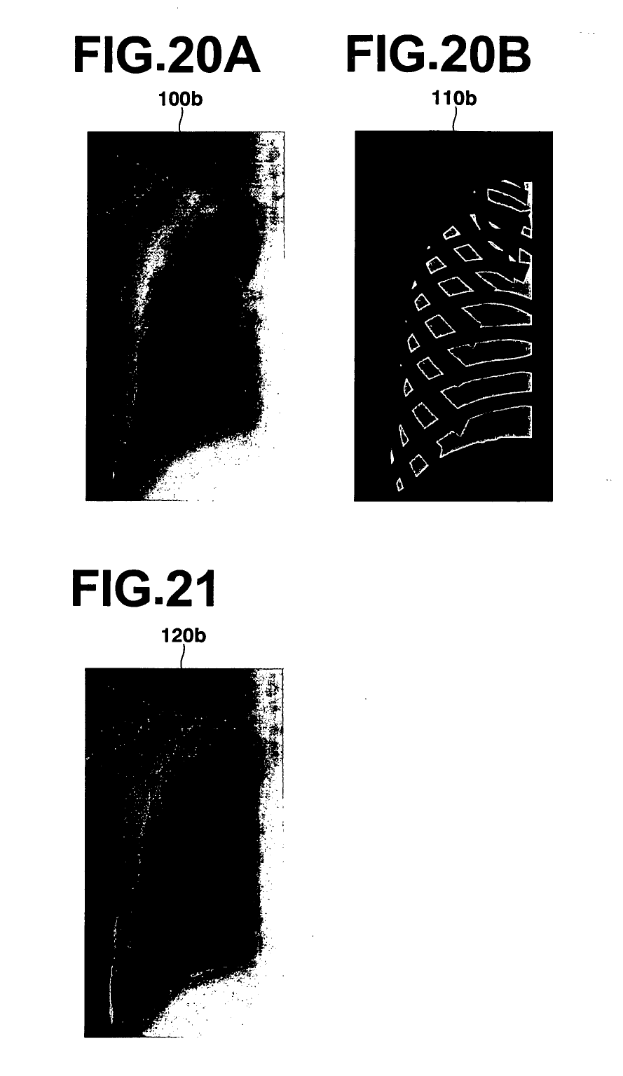

The non-rib region extraction means 30b then detects the lung field regions in the chest image 100b. More specifically, a method of automatic extraction of cardiothoracic outline can be used, as has been disclosed in Japanese Unexamined Patent Publication No. 2003-006661 proposed by the assignee. In this method, the chest image is converted into polar coordinates with reference to a point that is substantially the center of the cardiothoracic region, and template matching is carried out in a polar coordinate plane by use of a template having substantially the same shape as an average cardiothoracic outline, for automatic extraction of the cardiothoracic outline. The non-rib region excluding the rib region inferred by the rib region inference means 20b is extracted from the detected lung field regions (S2101). An image 110b of the non-rib region shown in FIG. 20B becomes the image of the soft tissues by removal of the rib region from the lung fields in the chest image 100b shown in FIG. 20A.

The soft tissue image inference means 40b artificially generates the inferred soft tissue image through inference of the image of the entire soft tissues from the image 110b of the non-rib region having been extracted (S2102). More specifically, the inferred soft tissue image is generated by using a result of statistical analysis of a plurality of soft tissue images obtained through energy subtraction. Principal component analysis is carried out as the analysis on the plurality of soft tissue images obtained by energy subtraction processing, for finding principal components (vector components) of the soft tissue images. The inferred soft tissue image can be reproduced artificially by use of the principal components.

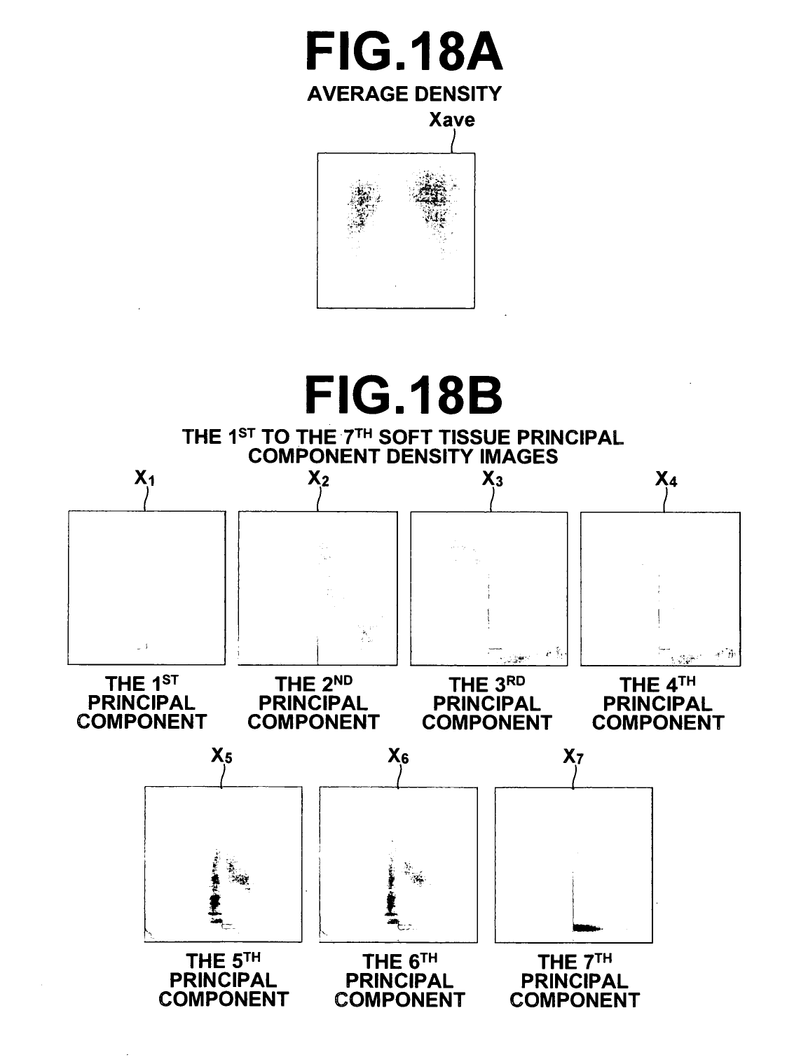

Firstly, each of the soft tissue images is transformed into a normalized shape such as a rectangle shown in FIGS. 18A and 18B.

Let coordinates before the transformation and corresponding coordinates after the transformation be B(x_{B}, y_{B}) and A(x_{A}, y_{A}), respectively. At the time of the transformation, the y-coordinate of B is converted into the corresponding y-coordinate of A according to Equation (1) below if the y-coordinate of the highest point and the lowest point in lung fields are represented by y_{B,up} and y_{B,down} for B and by y_{A,up} and y_{A,down} for A, respectively:

$$\begin{matrix}{y}_{A} = {y}_{A , up} + \frac{{y}_{A , down} - {y}_{A , up}}{{y}_{B , down} - {y}_{B , up}} ( {y}_{B} - {y}_{B , up} ) & \end{matrix} \qquad ( 1 )$$

Let positions of the right and left lung fields at y_{B} be represented by x_{B,left} and x_{B,right} while positions thereof at y_{A}be denoted by x_{A,left} and x_{A,right}. The transformation is carried out so as to cause the positions of the right and left lung fields at y_{B} to agree with the positions thereof at y_{A}according to Equation (2) below:

$${x}_{A} = {x}_{A , left} + \frac{{x}_{A , right} - {x}_{A , left}}{{x}_{B , right} - {x}_{B , left}} ( {x}_{B} - {x}_{B , left} ) \qquad ( 2 )$$

An average image Xₐᵥₑ (shown by FIG. 18A) of the soft tissue images having been transformed into the rectangular shape (hereinafter referred to as the average soft tissue density image) is found together with principal component soft tissue density images Xᵢ (i=1, 2, 3, . . . ) as the first to the n^{th} principal components (see FIG. 18B wherein the first to the seventh principal components are shown) obtained by principal component analysis on subtraction images between the soft tissue images and the average soft tissue density image. An inferred soft tissue image X can then be represented by Equation (3) below, by the average soft tissue density image Xₐᵥₑ and weighted addition of the principal component soft tissue density images Xᵢ:
X=Xₐᵥₑ+Σᵢaᵢ·Xᵢ (3)
where
X is a vector whose components are pixel values in the soft tissue image,
Xₐᵥₑ is a vector whose components are pixel values in the average soft tissue density image,
Xᵢ is a principal component vector representing the i^{th} principal component soft tissue density image, and
aᵢ is a weight coefficient for the i^{th} principal component vector.

For inferring the soft tissue image of the chest image 100b of the subject as the target pf examination, the weight coefficients are determined so as to cause the values of X to agree with pixel values of the non-rib region according to Equation (3). In this manner, an inferred soft tissue image 120b of the subject is generated as shown in FIG. 21.

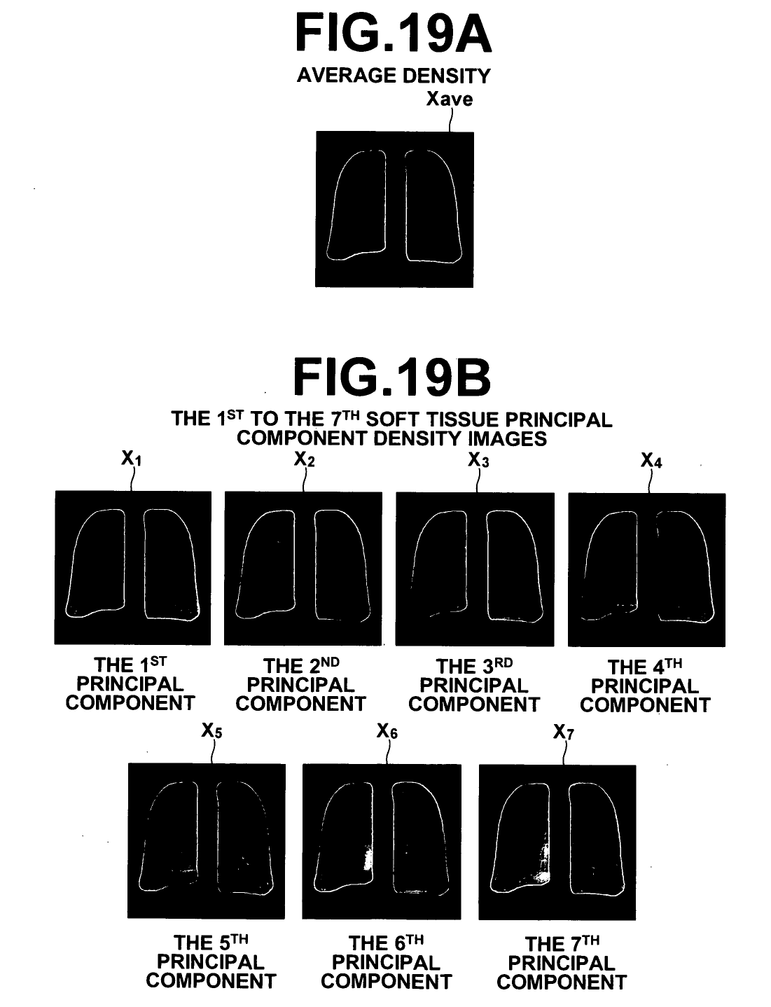

Alternatively, as shown in FIGS. 19A and 19B, the soft tissue images are normalized into an average soft tissue shape, and an average soft tissue density image (FIG. 19A) and principal component density images (FIG. 19B) are generated. Weight coefficients are determined so as to cause the values calculated by use of the weight coefficients to agree with density of the soft tissues other than the ribs in the chest image 100b of the subject, for generating the inferred soft tissue image X (shown in FIG. 21, for example) of the subject.

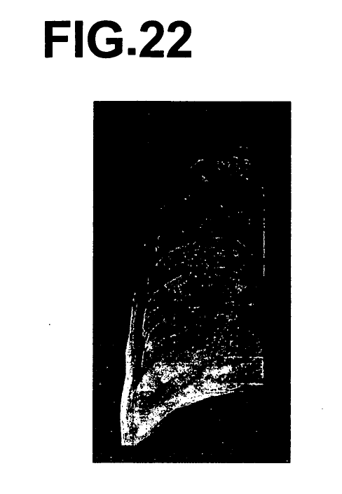

The inferred bone image generation means 50b removes the density contributing to the soft tissues in the chest image 100b by subtracting the inferred soft tissue image from the chest image 100b, for generating the inferred bone image shown in FIG. 22 based on pixel value components contributing to the ribs among the pixel values in the chest image 100b (S2103).

The rib region detection means 60b then recognizes the rib shapes in the inferred bone image by using an edge extraction filter and by using Huff transform or the like for detecting parabolic lines in the same manner as the rib region inference means 20b, and detects the rib region based on the rib shapes (S2104).

Alternatively, the rib region detection means 60b may extract rib shapes from chest images of a plurality of subjects so that model rib shapes M can be generated by use of a result of principal component analysis on the extracted rib shapes. The model rib shape M that is most similar to the rib shapes extracted from the chest image of the subject is searched for from among the model rib shapes M, and the model rib shape M having been found is inferred to be the rib shapes of the subject. Based on the rib shapes having been inferred, the rib region is detected.

More specifically, the rib shapes of the chest images are subjected to the principal component analysis in the following manner, for generating the model rib shapes.

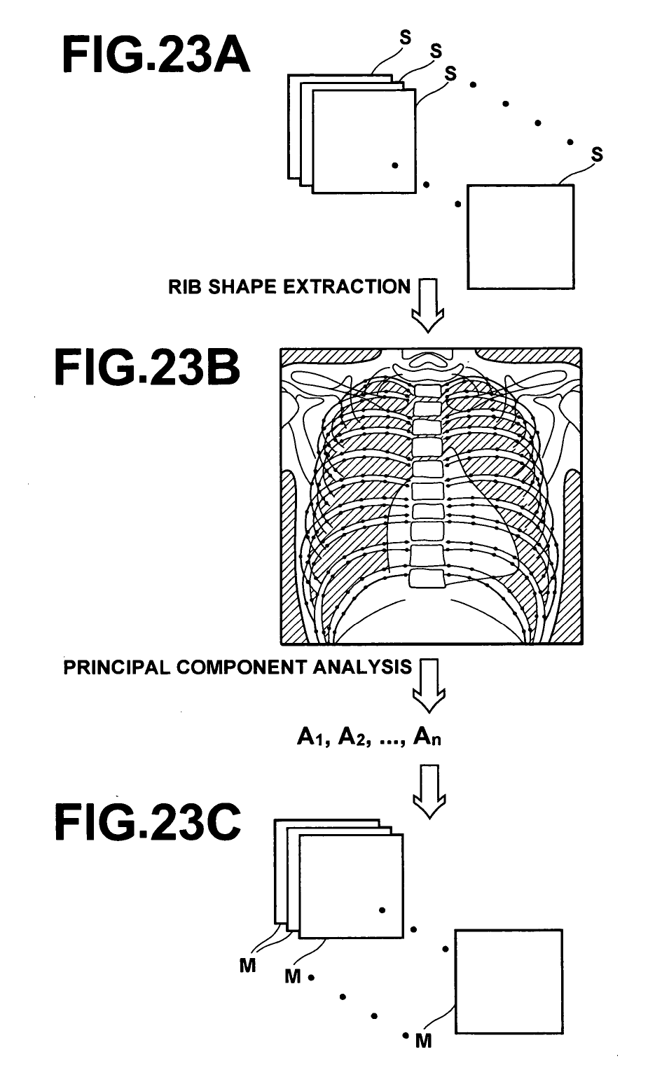

Firstly, rib shapes (shown in FIG. 23B) are detected in each of chest images S (shown in FIG. 23A) representing normal chests. The whole shape of the ribs is represented by points (referred to as characteristic points and shown by dots in FIG. 23B) forming outlines of the ribs. A shape vector X representing the entire shape of the ribs can be represented by Equation (5) below, by listing coordinates of 100 points extracted from the ribs:
X=(x₀,y₀,x₁,y₁, . . . ,x₉₉,y₉₉)^{T} (5)

Among the chest images S representing the normal chests radiographed in the past, the shape vectors X represented by Equation (5) above are extracted and subjected to principal component analysis for finding principal component vectors. In this manner, the rib shapes of the chest images S can be represented by a small number of independent vector components. More specifically, in the case where subtraction vectors between an average shape and the rib shapes of the respective chest images S are subjected to principal component analysis for obtaining the first to the nth principal component vectors Aᵢ (i=1, 2, . . . , n), the model rib shapes M can be represented by Equation (6) below by use of an average rib shape vector and the principal component vectors Aᵢ shown in FIG. 23C:

$$M = {X}_{b} + {\sum }_{i}^{} {α}_{i} {A}_{i} \qquad ( 6 )$$

where X_{b} is an average rib shape vector and αᵢ is a weight coefficient for the i^{th} principal component vector.

By changing the weight coefficients in Equation (6), the model rib shapes M are generated, and the model rib shape M closest to the radiographed rib shapes of the subject is selected among the model rib shapes M. Based on the rib shapes thereof, the rib region is detected.

The rib region detected in the chest image wherein the soft tissues have been removed becomes more accurate than the rib region inferred by the rib region inference means 20b.

As has been described above, since the rib region is detected by removing a background image including the soft tissue image, the rib region can be detected accurately. If the ribs are removed from the original image by using the rib image generated in this manner, the soft tissue image can be extracted with accuracy, which enables accurate detection of an abnormal shadow caused by cancer or the like.

The rib region inference means 20b may infer the rib region according to the method of principal component analysis adopted by the rib region detection means 60b.

By installing a program having the means described above in a computer, the computer can function as the image processing apparatus.

1. image processing apparatus comprising:
chest image storage means for storing chest images obtained by plain radiography of the chests of a plurality of subjects;
rib image generation means for generating rib images by extracting pixel value components contributing to ribs from values of pixels comprising the respective chest images;
rib overlap detection means for detecting rib overlaps where the ribs appear to overlap in rib regions in the respective rib images generated by the rib image generation means;
image normalization means for normalizing the rib images so as to cause positions of the rib overlaps detected by the rib overlap detection means in the respective chest images to agree in all the rib images;
rib image analysis means for carrying out analysis on pixel values of the rib images by applying a statistical method to the rib images having been normalized; and
rib image inference means for generating an inferred rib image by inferring pixel values of ribs in a chest image obtained by radiography of a predetermined subject, by using a result of the analysis by the rib image analysis means.

2. The image processing apparatus according to claim 1 further comprising image division means for dividing the ribs in the respective rib images into partial rib images individually representing the respective ribs, wherein
the image normalization means normalizes the partial rib images by transformation thereof so as to cause the positions of the rib overlaps in the partial rib images corresponding each other to agree, after transforming the partial rib images into a predetermined normalized shape.

3. The image processing apparatus according to claim 2, wherein the rib image analysis means obtains principal component images by carrying out principal component analysis on the pixel values of the rib images of the respective subjects and
the rib image inference means generates the inferred rib image by inferring the pixel values of the ribs of the predetermined subject through weighted addition of the principal component images.

4. The image processing apparatus according to claim 1 wherein the rib image inference means generates a rib image by extracting pixel value components contributing to the ribs from pixel values comprising the chest image of the predetermined subject and infers pixel values of normal ribs of the subject from at least a part of the rib image.

5. An image processing apparatus comprising:
chest image storage means for storing a chest image obtained by plain radiography of the chest of a subject;
rib image generation means for generating a rib image by extracting pixel value components contributing to ribs from values of pixels comprising the chest image;
rib shape extraction means for extracting shapes of the respective ribs from the chest image or the rib image;
model rib shape setting means for setting a model rib shape corresponding to an anatomical rib structure along the shape of each of the ribs, based on the shape thereof among the shapes extracted by the rib shape extraction means and pixel values in a region thereof in the rib image; and
rib image inference means for generating an inferred rib image by inferring pixel values of the respective ribs in the chest image, based on the model rib shapes set by the model rib shape setting means.

6. The image processing apparatus according to claim 5 wherein the model rib shapes are tube-like shapes along long axes of the respective rib shapes.

7. An image processing apparatus comprising:
chest image storage means for storing a chest image obtained by plain radiography of the chest of a subject;
rib region inference means for inferring a rib region in the chest image;
non-rib region extraction means for extracting a non-rib region as a region other than the rib region from lung field regions in the chest image;
soft tissue image inference means for generating an inferred soft tissue image by inferring pixel value components contributing to soft tissues in the lung field regions in the chest image, based on an image of the non-rib region;
inferred bone image generation means for generating an inferred bone image comprising pixel value components contributing to ribs among pixel values of the chest image, through removal of the inferred soft tissue image from the chest image; and
rib region detection means for detecting a rib region in the inferred bone image.

8. The image processing apparatus according to claim 7 wherein the soft tissue image inference means generates the inferred soft tissue image based on a result of analysis on pixel values of soft tissues in chest images obtained by radiography of a large number of subjects, by use of statistical analysis means.

9. The image processing apparatus according to claim 8 wherein the analysis means is principal component analysis and obtains principal component images of the soft tissues as a result of the analysis and
the soft tissue image inference means generates the inferred soft tissue image by inferring pixel values of the soft tissues of the predetermined subject through weighted addition of the principal component images.

10. An image processing method comprising the steps of:
storing chest images obtained by plain radiography of the chests of a plurality of subjects in chest image storage means;
generating rib images by extracting pixel value components contributing to ribs from values of pixels comprising the respective chest images;
detecting rib overlaps where the ribs appear to overlap in rib regions in the respective rib images generated by the rib image generating step;
normalizing the rib images so as to cause positions of the rib overlaps detected by the rib overlap detecting step in the respective chest images to agree in all the rib images;
carrying out analysis on pixel values of the rib images by applying a statistical method to the rib images having been normalized; and
generating an inferred rib image by inferring pixel values of ribs in a chest image obtained by radiography of a predetermined subject, by using a result of the analysis.

11. An image processing method comprising the steps of:
storing a chest image obtained by plain radiography of the chest of a subject in chest image storage means;
generating a rib image by extracting pixel value components contributing to ribs from values of pixels comprising the chest image;
extracting shapes of the respective ribs from the chest image or the rib image;
setting a model rib shape corresponding to an anatomical rib structure along the shape of each of the ribs, based on the shape thereof among the shapes extracted by the rib shape extracting step and pixel values in a region thereof in the rib image; and
generating an inferred rib image by inferring pixel values of the respective ribs in the chest image, based on the model rib shapes set by the model rib shape setting step.

12. An image processing method comprising the steps of:
storing a chest image obtained by plain radiography of the chest of a subject in chest image storage means; inferring a rib region in the chest image;
extracting a non-rib region as a region other than the rib region from lung field regions in the chest image;
generating an inferred soft tissue image by inferring pixel value components contributing to soft tissues in the lung field regions in the chest image, based on an image of the non-rib region;
generating an inferred bone image comprising pixel value components contributing to ribs among pixel values of the chest image, through removal of the inferred soft tissue image from the chest image; and detecting a rib region in the inferred bone image.

13. A program causing a computer to function as:
chest image storage means for storing chest images obtained by plain radiography of the chests of a plurality of subjects;
rib image generation means for generating rib images by extracting pixel value components contributing to ribs from values of pixels comprising the respective chest images;
rib overlap detection means for detecting rib overlaps where the ribs appear to overlap in rib regions in the respective rib images generated by the rib image generation means;
image normalization means for normalizing the rib images so as to cause positions of the rib overlaps detected by the rib overlap detection means in the respective chest images to agree in all the rib images;
rib image analysis means for carrying out analysis on pixel values of the rib images by applying a statistical method to the rib images having been normalized; and
rib image inference means for generating an inferred rib image by inferring pixel values of ribs in a chest image obtained by radiography of a predetermined subject, by using a result of the analysis by the rib image analysis means.

14. A program causing a computer to function as:
chest image storage means for storing a chest image obtained by plain radiography of the chest of a subject;
rib image generation means for generating a rib image by extracting pixel value components contributing to ribs from values of pixels comprising the chest image;
rib shape extraction means for extracting shapes of the respective ribs from the chest image or the rib image;
model rib shape setting means for setting a model rib shape corresponding to an anatomical rib structure along the shape of each of the ribs, based on the shape thereof among the shapes extracted by the rib shape extraction means and pixel values in a region thereof in the rib image; and
rib image inference means for generating an inferred rib image by inferring pixel values of the respective ribs in the chest image, based on the model rib shapes set by the model rib shape setting means.

15. A program causing a computer to function as:
chest image storage means for storing a chest image obtained by plain radiography of the chest of a subject;
rib region inference means for inferring a rib region in the chest image;
non-rib region extraction means for extracting a non-rib region as a region other than the rib region from lung field regions in the chest image;
soft tissue image inference means for generating an inferred soft tissue image by inferring pixel value components contributing to soft tissues in the lung field regions in the chest image, based on an image of the non-rib region;
inferred bone image generation means for generating an inferred bone image comprising pixel value components contributing to ribs among pixel values of the chest image, through removal of the inferred soft tissue image from the chest image; and
rib region detection means for detecting a rib region in the inferred bone image.
